(19) United States
(12) Patent Application Publication
(10) Pub. No.: US 2026/0083973 A1
(43) Pub. Date: Mar. 26, 2026

(54) Automated Optimization of Deep Brain Stimulation (DBS) Parameters Using Weighted Targets and Avoidance Regions

(71) Applicant: Boston Scientific Neuromodulation Corporation, Valencia, CA (US)

(72) Inventors: Mahsa Malekmohammadi, Sherman Oaks, CA (US); Richard Mustakos, Simi Valley, CA (US); Stephen Carcieri, Los Angeles, CA (US); Peter Yoo, Burbank, CA (US)

(21) Appl. No.: 19/299,166

(22) Filed: Aug. 13, 2025

Related U.S. Application Data
(60) Provisional application No. 63/699,033, filed on Sep. 25, 2024.

(51) Int. Cl. A61N 1/372; A61N 1/05; A61N 1/36

(52) U.S. Cl. CPC A61N 1/37264; A61N 1/0534; A61N 1/36064; A61N 1/36067; A61N 1/36096; A61N 1/3615; A61N 1/37247

(57) ABSTRACT

Methods and systems for assisting the programming of stimulation parameters for deep brain stimulation (DBS) for a patient are described. Target regions for stimulation and avoidance regions where stimulation is to be avoided can be identified and weights can be associated with the target and avoidance regions. The described methods and systems use the weights to optimize stimulation.

### CROSS REFERENCE TO RELATED APPLICATIONS

This is a non-provisional application of U.S. Provisional Patent Application Ser. No. 63/699,033, filed Sep. 25, 2024, which is incorporated herein by reference in its entirety, and to which priority is claimed.

### FIELD OF THE INVENTION

This application relates to deep brain stimulation (DBS), and more particularly, to methods and systems for optimizing DBS.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Deep Brain Stimulation (DBS) context. DBS has been applied therapeutically for the treatment of neurological disorders, including Parkinson's Disease, essential tremor, dystonia, and epilepsy, to name but a few. Further details discussing the treatment of diseases using DBS are disclosed in U.S. Pat. Nos. 6,845,267, and 6,950,707.

Each of these neurostimulation systems, whether implantable or external, typically includes one or more electrode-carrying stimulation leads, which are implanted at the desired stimulation site, and a neurostimulator, used externally or implanted remotely from the stimulation site, but coupled either directly to the neurostimulation lead(s) or indirectly to the neurostimulation lead(s) via a lead extension. The neurostimulation system may further comprise a handheld external control device to remotely instruct the neurostimulator to generate electrical stimulation pulses in accordance with selected stimulation parameters. Typically, the stimulation parameters programmed into the neurostimulator can be adjusted by manipulating controls on the external control device to modify the electrical stimulation provided by the neurostimulator system to the patient.

Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

Non-optimal electrode placement and stimulation parameter selections may result in excessive energy consumption due to stimulation that is set at too high amplitude, too wide a pulse duration, or too fast a frequency; inadequate or marginalized treatment due to stimulation that is set at too low an amplitude, too narrow a pulse duration, or too slow a frequency; or stimulation of neighboring cell populations that may result in undesirable side effects. For example, bilateral DBS of the subthalamic nucleus (STN) has been shown to provide effective therapy for improving the major motor signs of advanced Parkinson's disease, and although the bilateral stimulation of the subthalamic nucleus is considered safe, an emerging concern is the potential negative consequences that it may have on cognitive functioning and overall quality of life (see A. M. M. Frankemolle, et al., Reversing Cognitive-Motor Impairments in Parkinson's Disease Patients Using a Computational Modelling Approach to Deep Brain Stimulation Programming, Brain 2010; pp. 1-16). In large part, this phenomenon is due to the small size of the STN. Even with the electrodes located predominately within the sensorimotor territory, the electrical field generated by DBS is non-discriminately applied to all neural elements surrounding the electrodes, thereby resulting in the spread of current to neural elements affecting cognition. As a result, diminished cognitive function during stimulation of the STN may occur due to non-selective activation of non-motor pathways within or around the STN.

The large number of electrodes available, combined with the ability to generate a variety of complex stimulation pulses, presents a huge selection of stimulation parameter sets to the clinician or patient. In the context of DBS, neurostimulation leads with a complex arrangement of electrodes that not only are distributed axially along the leads, but are also distributed circumferentially around the neurostimulation leads as segmented electrodes, can be used.

To facilitate such selection, the clinician generally programs the external control device, and if applicable the neurostimulator, through a computerized programming system. This programming system can be a self-contained hardware/software system, or can be defined predominantly by software running on a standard personal computer (PC) or mobile platform. The PC or custom hardware may actively control the characteristics of the electrical stimulation generated by the neurostimulator to allow the optimum stimulation parameters to be determined based on patient feedback and to subsequently program the external control device with the optimum stimulation parameters.

When electrical leads are implanted within the patient, the computerized programming system may be used to instruct the neurostimulator to apply electrical stimulation to test placement of the leads and/or electrodes, thereby assuring that the leads and/or electrodes are implanted in effective locations within the patient. The system may also instruct the user how to improve the positioning of the leads or confirm when a lead is well-positioned. Once the leads are correctly positioned, a fitting procedure, which may be referred to as a navigation session, may be performed using the computerized programming system to program the external control device, and if applicable the neurostimulator, with a set of stimulation parameters that best addresses the neurological disorder(s). There is a need for methods and systems that assist a clinician in determining optimum stimulation parameters for treating the patient.

### SUMMARY

Disclosed herein is a method executed on an external programmer for programming a pulse generator (PG) for providing deep brain stimulation (DBS) to a patient having one or more electrode leads implanted in the patient's brain, wherein each electrode lead comprises a plurality of electrodes, the method comprising: receiving from a user interface (UI) of the external programmer an indication of at least one target region in the patient's brain to be stimulated, receiving from the UI an indication of two or more avoidance regions in the patient's brain for which stimulation is to be preferentially avoided, receiving from the UI an indication of weights assigned to each of the two or more avoidance regions, wherein at least two of the weights are different, for each of a plurality of trial stimulation parameter sets, using control circuitry of the external programmer to: determine a volume of the at least one target region that will be stimulated by electrical stimulation using the trial parameter set, determine a volume of each of the avoidance regions that will be stimulated by electrical stimulation using the trial parameter set, determine a metric based the volumes and the weights, using the control circuitry to use the metrics to select a therapeutic stimulation parameter set from the plurality of trial stimulation parameter sets, and using the control circuitry to program the PG with the therapeutic stimulation parameter set. According to some embodiments, determining a volume of the at least one target region that will be stimulated and the volume of each of the avoidance regions that will be stimulated comprises: determining a stimulation field model (SFM) for the trial stimulation set, wherein the SFM indicates of a volume of tissue activated (VTA) by the electrical stimulation using the parameter set, and determining a volume of overlap of the SFM with each of the one or more target regions and each of the two or more avoidance regions. According to some embodiments, the metric comprises a value determined by: (i) determining a weighted overlap for each of the two or more avoidance regions, each weighted overlap comprising the volume of overlap of the SFM with the avoidance region multiplied by the avoidance region's assigned weight, (ii) summing the weighted overlaps, and (iii) subtracting a summed overlaps from the volume of overlap of the SFM with one or more target regions. According to some embodiments, the metric is determined using the formula:

m=Σ(vt−Σ(va,i*wa,i)−(vSFM*wB))
wherein m is the metric value, vt is the overlap of the SFM with the one or more target regions, va, i is a volume of overlap of the SFM with an ith avoidance region, wa, i is the weight assigned to the ith avoidance region, vSFM is a total volume of the SFM and wB is a weight assigned to vSFM. According to some embodiments, determining the volume of overlap of the SFM with each of the one or more target regions and each of the two or more avoidance regions comprises: voxelizing the SFM, determining 3-dimensional models for each of the one or more target regions and each of the two or more avoidance regions, voxelizing the 3-dimensional models, and identifying a number of voxels that are common with the voxelized SFM and each of the one or more target regions and each of the two or more avoidance regions. According to some embodiments, each of the 3-dimensional models are based on imaging of the patient's brain. According to some embodiments, each of the parameter sets comprises a current fractionalization comprising a unique arrangement of current driven to each of the plurality of electrodes. According to some embodiments, each of the two or more avoidance regions are regions, the electrical stimulation of which, are associated with side effects. According to some embodiments, the weight assigned to each of the avoidance regions is indicative of a criticality of avoiding stimulation of the respective avoidance region. According to some embodiments, the method further comprises: receiving from the UI an indication of one or more stimulation criteria, and using the control circuitry to use the one or more stimulation criteria, along with the one or more target regions and the two or more avoidance regions, to select the therapeutic stimulation parameter set. According to some embodiments, the one or more stimulation criteria are selected from the group consisting of a threshold value of one or more stimulation parameters, a total charge injected into the tissue during stimulation, a charge injected into the tissue per stimulation pulse or period, a total energy delivered, a total amplitude, a maximum power usage, and a total volume of the stimulated tissue. According to some embodiments, using the control circuitry to use the one or more stimulation criteria, along with the one or more target regions and the two or more avoidance regions, to select the therapeutic stimulation parameter set comprises eliminating parameter sets that do not meet the stimulation criteria from the plurality of trial stimulation parameter sets. According to some embodiments, the method further comprises receiving from the UI an indication of weights assigned to each of the one or more stimulation criteria. According to some embodiments, the metric is further determined based on the one or more stimulation criteria their respective weights. According to some embodiments, the therapeutic parameter set is configured to treat one or more of Parkinson's disease, depression (e.g., treatment-resistant depression), essential tremor, dystonia, epilepsy, and/or obsessive-compulsive disorder. According to some embodiments, the method further comprises displaying one or more selection elements on the UI, whereby a user may select the at least one target region and the two or more avoidance regions. According to some embodiments, the method further comprises displaying one or more selection elements on the UI, whereby a user may assign weights to the at least one target region and the two or more avoidance regions. According to some embodiments, the method further comprises using the UI to display one or more images representative of the one or more target regions and the at least two avoidance regions. According to some embodiments, the control circuitry is configured to form the images from one or more of pre-operative and/or post-operative tissue imaging. According to some embodiments, the method further comprises using the UI to display a representation of the one or more electrode leads overlaid with the images representative of the one or more target regions and the at least two avoidance regions. According to some embodiments, selecting a therapeutic stimulation parameter set comprises using a reverse programming algorithm. According to some embodiments, the reverse programming algorithm comprises optimizing a current fractionalization among the electrode contacts based on stimulation field models (SFMs) modeled for each of the trial stimulation parameter sets. According to some embodiments, the reverse programming algorithm comprises a cost function that includes (i) overlap of the SFMs with the target structure for each current fractionalization, and (ii) a cost associated with increasing a size of the SFM. According to some embodiments, the cost function is further a function of (iii) overlap of the SFMs with an avoidance structure for each current fractionalization.

Also disclosed herein is an external programmer for programming a pulse generator (PG) for providing deep brain stimulation (DBS) to a patient having one or more electrode leads implanted in the patient's brain, wherein each electrode lead comprises a plurality of electrodes, the external programmer comprising: control circuitry configured to: receive from a user interface (UI) of the external programmer an indication of at least one target region in the patient's brain to be stimulated, receive from the UI an indication of two or more avoidance regions in the patient's brain for which stimulation is to be preferentially avoided, receive from the UI an indication of weights assigned to each of the two or more avoidance regions, wherein at least two of the weights are different, for each of a plurality of trial stimulation parameter sets: determine a volume of the at least one target region that will be stimulated by electrical stimulation using the trial parameter set, determine a volume of each of the avoidance regions that will be stimulated by electrical stimulation using the trial parameter set, determine a metric based the volumes and the weights, use the metrics to select a therapeutic stimulation parameter set from the plurality of trial stimulation parameter sets, and program the PG with the therapeutic stimulation parameter set. According to some embodiments, determining a volume of the at least one target region that will be stimulated and the volume of each of the avoidance regions that will be stimulated comprises: determining a stimulation field model (SFM) for the trial stimulation set, wherein the SFM indicates of a volume of tissue activated (VTA) by the electrical stimulation using the parameter set, and determining a volume of overlap of the SFM with each of the one or more target regions and each of the two or more avoidance regions. According to some embodiments, the metric comprises a value determined by: (i) determining a weighted overlap for each of the two or more avoidance regions, each weighted overlap comprising the volume of overlap of the SFM with the avoidance region multiplied by the avoidance region's assigned weight, (ii) summing the weighted overlaps, and (iii) subtracting a summed overlaps from the volume of overlap of the SFM with one or more target regions. According to some embodiments, the metric is determined using the formula:

$$m = \sum ( vt - \sum ( va , i * wa , i ) - ( vSFM * wB ) )$$

wherein m is the metric value, vt is the overlap of the SFM with the one or more target regions, va, i is a volume of overlap of the SFM with an ith avoidance region, wa, i is the weight assigned to the ith avoidance region, vSFM is a total volume of the SFM and wB is a weight assigned to vSFM. According to some embodiments, determining the volume of overlap of the SFM with each of the one or more target regions and each of the two or more avoidance regions comprises: voxelizing the SFM, determining 3-dimensional models for each of the one or more target regions and each of the two or more avoidance regions, voxelizing the 3-dimensional models, and identifying a number of voxels that are common with the voxelized SFM and each of the one or more target regions and each of the two or more avoidance regions. According to some embodiments, each of the 3-dimensional models are based on imaging of the patient's brain. According to some embodiments, each of the parameter sets comprises a current fractionalization comprising a unique arrangement of current driven to each of the plurality of electrodes. According to some embodiments, each of the two or more avoidance regions are regions, the electrical stimulation of which, are associated with side effects. According to some embodiments, the weight assigned to each of the avoidance regions is indicative of a criticality of avoiding stimulation of the respective avoidance region. According to some embodiments, the control circuitry is further configured to: receive from the UI an indication of one or more stimulation criteria, and use the control circuitry to use the one or more stimulation criteria, along with the one or more target regions and the two or more avoidance regions, to select the therapeutic stimulation parameter set. According to some embodiments, the one or more stimulation criteria are selected from the group consisting of a threshold value of one or more stimulation parameters, a total charge injected into the tissue during stimulation, a charge injected into the tissue per stimulation pulse or period, a total energy delivered, a total amplitude, a maximum power usage, and/or a total volume of the stimulated tissue. According to some embodiments, using the control circuitry to use the one or more stimulation criteria, along with the one or more target regions and the two or more avoidance regions, to select the therapeutic stimulation parameter set comprises eliminating parameter sets that do not meet the stimulation criteria from the plurality of trial stimulation parameter sets. According to some embodiments, the control circuitry is further configured to receive from the UI an indication of weights assigned to each of the one or more stimulation criteria. According to some embodiments, the metric is further determined based on the one or more stimulation criteria their respective weights. According to some embodiments, the therapeutic parameter set is configured to treat one or more of Parkinson's disease, depression (e.g., treatment-resistant depression), essential tremor, dystonia, epilepsy, and/or obsessive-compulsive disorder. According to some embodiments, the external programmer is further configured to display one or more selection elements on the UI, whereby a user may select the at least one target region and the two or more avoidance regions. According to some embodiments, the external programmer is further configured to display one or more selection elements on the UI, whereby a user may assign weights to the at least one target region and the two or more avoidance regions. According to some embodiments, the external programmer is further configured to use the UI to display one or more images representative of the one or more target regions and the at least two avoidance regions. According to some embodiments, the control circuitry is configured to form the images from one or more of pre-operative and/or post-operative tissue imaging. According to some embodiments, the external programmer is further configured to use the UI to display a representation of the one or more electrode leads overlaid with the images representative of the one or more target regions and the at least two avoidance regions. According to some embodiments, selecting a therapeutic stimulation parameter set comprises using a reverse programming algorithm. According to some embodiments, the reverse programming algorithm comprises optimizing a current fractionalization among the electrode contacts based on stimulation field models (SFMs) modeled for each of the trial stimulation parameter sets. According to some embodiments, the reverse programming algorithm comprises a cost function that includes (i) overlap of the SFMs with the target structure for each current fractionalization, and (ii) a cost associated with increasing a size of the SFM. According to some embodiments, the cost function is further a function of (iii) overlap of the SFMs with an avoidance structure for each current fractionalization.

Also disclosed herein is a method for programming a pulse generator (PG) for providing deep brain stimulation (DBS) to a patient having one or more electrode leads implanted in the patient's brain, wherein each electrode lead comprises a plurality of electrodes, the method comprising: determining at least a first and a second target region in the patient's brain, each to be stimulated, for each of a plurality of trial stimulation parameter sets: determining a volume of the at least the first and of the second target region that will be stimulated by a stimulation field generated by electrical stimulation using the trial parameter set, determining a metric, wherein the metric is based on the volumes and on a cost parameter, using the metrics to select a best stimulation parameter set, and programming the PG with the best stimulation parameter set. According to some embodiments, the cost parameter comprises a ratio of a cost of stimulating at least one defined region of tissue over a benefit of stimulating regions of the first and second targets. According to some embodiments, determining a volume of the at least the first and second target regions that will be stimulated comprises: determining a stimulation field model (SFM) for the trial stimulation set, wherein the SFM indicates of a volume of tissue activated (VTA) by the electrical stimulation using the parameter set, and determining a volume of overlap of the SFM with each of the at least the first and second target regions. According to some embodiments, selecting the best stimulation parameter set comprises using a reverse programming algorithm. According to some embodiments, the reverse programming algorithm comprises optimizing a current fractionalization among the electrode contacts based on stimulation field models (SFMs) modeled for each of the trial stimulation parameter sets. According to some embodiments, the cost function includes (i) overlap of the SFMs with the target structures for each current fractionalization, and (ii) a cost associated with increasing a size of the SFM, and wherein the reverse programming algorithm minimizes the cost function. According to some embodiments, the cost function is further a function of (iii) overlap of the SFMs with an avoidance structure for each current fractionalization.

Also disclosed herein is an external programmer for programming a pulse generator (PG) for providing deep brain stimulation (DBS) to a patient having one or more electrode leads implanted in the patient's brain, wherein each electrode lead comprises a plurality of electrodes, the external programmer comprising: control circuitry configured to execute a method comprising: determining at least a first and a second target region in the patient's brain, each to be stimulated, for each of a plurality of trial stimulation parameter sets: determining a volume of the at least the first and of the second target region that will be stimulated by a stimulation field generated by electrical stimulation using the trial parameter set, determining a metric, wherein the metric is based on the volumes and on a cost parameter, using the metrics to select a best stimulation parameter set, and programming the PG with the best stimulation parameter set. According to some embodiments, the cost parameter comprises a ratio of a cost of stimulating at least one defined region of tissue over a benefit of stimulating regions of the first and second targets. According to some embodiments, determining a volume of the at least the first and second target regions that will be stimulated comprises: determining a stimulation field model (SFM) for the trial stimulation set, wherein the SFM indicates of a volume of tissue activated (VTA) by the electrical stimulation using the parameter set, and determining a volume of overlap of the SFM with each of the at least the first and second target regions. According to some embodiments, selecting the best stimulation parameter set comprises using a reverse programming algorithm. According to some embodiments, the reverse programming algorithm comprises optimizing a current fractionalization among the electrode contacts based on stimulation field models (SFMs) modeled for each of the trial stimulation parameter sets. According to some embodiments, the cost function includes (i) overlap of the SFMs with the target structures for each current fractionalization, and (ii) a cost associated with increasing a size of the SFM, and wherein the reverse programming algorithm minimizes the cost function. According to some embodiments, the cost function is further a function of (iii) overlap of the SFMs with an avoidance structure for each current fractionalization.

Also disclosed herein is a method for programming a pulse generator (PG) for providing deep brain stimulation (DBS) to a patient having one or more electrode leads implanted in the patient's brain, wherein each electrode lead comprises a plurality of electrodes, the method comprising: determining at least a first and a second target region in the patient's brain, each to be stimulated, determining a first best trial parameter set for the first target region, determining a second best trial parameter set for the second target region, combining the first and second best trial parameter sets to yield a best stimulation parameter set to stimulate bot the first and second target regions, and programming the PG with the best stimulation parameter set. According to some embodiments, each of the best trial stimulation parameter sets comprises a current fractionalization comprising a unique arrangement of current driven to each of the plurality of electrodes and an amplitude, and wherein combining the first and second best trial parameter sets comprises: determining a first vector defined by the current fractionalization and amplitude of first best trial parameter set, determining a second vector defined by the current fractionalization and amplitude of second best trial parameter set, and adding the first and second vectors. According to some embodiments, determining the first and second best trial parameter sets each comprise: for each of a plurality of trial stimulation parameter sets: determining a volume of the at least the first or of the second target region that will be stimulated by a stimulation field generated by electrical stimulation using the trial parameter set, determining a metric, wherein the metric is based on the volume and on a cost parameter, and using the metrics to select the best trial stimulation parameter set.

The invention may also reside in the form of a programed external device, such as a clinician programmer or other computing device (via its control circuitry) for carrying out the above methods, a programmed implantable pulse generator (IPG) or external trial stimulator (ETS) (via its control circuitry) for carrying out the above methods, a system including a programmed external device and IPG or ETS for carrying out the above methods, or as a computer-readable media for carrying out the above methods stored in an external device or IPG or ETS. The invention may also reside in one or more non-transitory computer-readable media comprising instructions, which when executed by a processor of a machine configure the machine to perform any of the above methods.

### BRIEF DESCRIPTION OF THE DRAWINGS

FIG. 1A shows an Implantable Pulse Generator (IPG).

FIG. 1B shows a percutaneous lead having split-ring electrodes.

FIGS. 2A and 2B show an example of stimulation pulses (waveforms) producible by the IPG or by an External Trial Stimulator (ETS).

FIG. 3 shows an example of stimulation circuitry useable in the IPG or ETS.

FIG. 4 shows an ETS environment useable to provide stimulation before implantation of an IPG.

FIG. 5 shows various external devices capable of communicating with and programming stimulation in an IPG or ETS.

FIG. 6 illustrates a generalized embodiment of a user interface (UI) for programming stimulation.

FIG. 7 illustrates an embodiment of a workflow for optimizing DBS parameters.

FIG. 8 illustrates an embodiment of a graphical user interface (GUI) for identifying and weighting target and avoidance regions.

FIG. 9 illustrates a stimulation field model (SFM) overlayed with selected brain regions.

### DETAILED DESCRIPTION

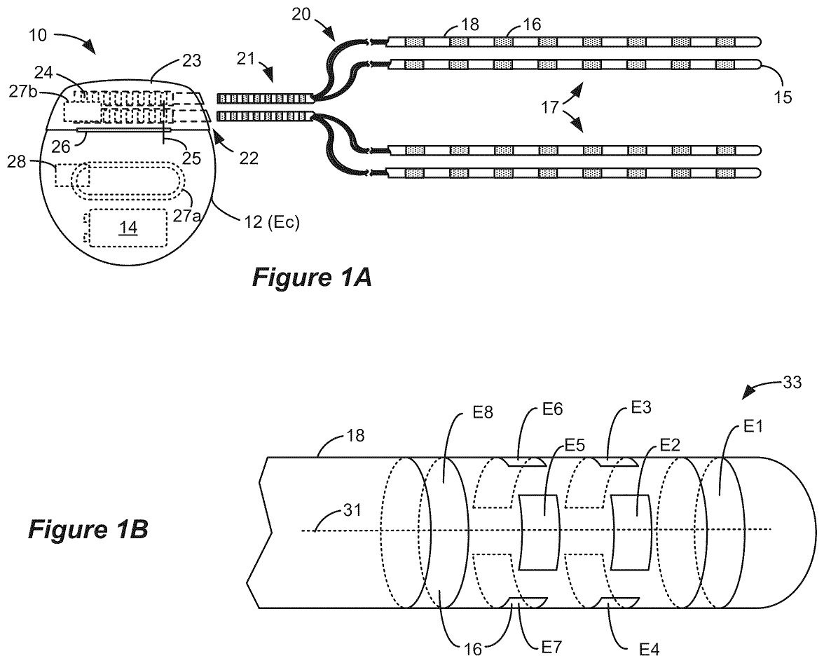

A DBS system typically includes an Implantable Pulse Generator (IPG), such as IPG 10 shown in FIG. 1A. The IPG 10 includes a biocompatible device case 12 that holds the circuitry and a battery 14 for providing power for the IPG to function. The IPG 10 is coupled to tissue-stimulating electrodes 16 via one or more electrode leads that form an electrode array 17. For example, one or more electrode leads 15 can be used having ring-shaped electrodes 16 carried on a flexible body 18.

In yet another example shown in FIG. 1B, an electrode lead 33 can include one or more split-ring directional electrodes. In this example, eight electrodes 16 (E1-E8) are shown. Electrode E1 at the distal end of the lead and electrode E8 at a proximal end of the lead comprise ring electrodes spanning 360 degrees around a central axis 31 of the lead 33. In some embodiments, the electrode E1 may be a “bullet tip” electrode, meaning that it can cover the tip of the electrode lead. Electrodes E2, E3, and E4 comprise split-ring electrodes, each of which are located at the same longitudinal position along the central axis 31, but with each spanning less than 360 degrees around the axis. For example, each of electrodes E2, E3, and E4 may span 90 degrees around the axis 31, with each being separated from the others by gaps of 30 degrees. Electrodes E5, E6, and E7 also comprise split-ring electrodes, but are located at a different longitudinal position along the central axis 31 than are split ring electrodes E4, E2, and E3. As shown, the split-ring electrodes E2-E4 and E5-E7 may be located at longitudinal positions along the axis 31 between ring electrodes E1 and E8. However, this is just one example of a lead 33 having split-ring electrodes. In other designs, all electrodes can be split-ring, or there could be different numbers of split-ring electrodes at each longitudinal position (i.e., more or less than three), or the ring and split-ring electrodes could occur at different or random longitudinal positions, etc.

Lead wires 20 within the leads are coupled to the electrodes 16 and to proximal contacts 21 insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the IPG 10 illustrated in FIG. 1A, there are thirty-two electrodes (E1-E32), split between four percutaneous leads 15, and thus the header 23 may include a 2×2 array of eight-electrode lead connectors 22. However, the type and number of leads, and the number of electrodes, in an IPG is application-specific and therefore can vary. The conductive case 12 can also comprise an electrode (Ec).

In a DBS application, as is useful in the treatment of tremor in Parkinson's disease for example, the IPG 10 is typically implanted under the patient's clavicle (collarbone). Lead wires 20 are tunneled through the neck and the scalp and the electrode leads 15 (or 33) are implanted through holes drilled in the skull and positioned for example in the subthalamic nucleus (STN).

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1A, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Bluetooth Low Energy (BLE), as described in U.S. Patent Publication 2019/0209851, Zigbee, WiFi, MICS, and the like.

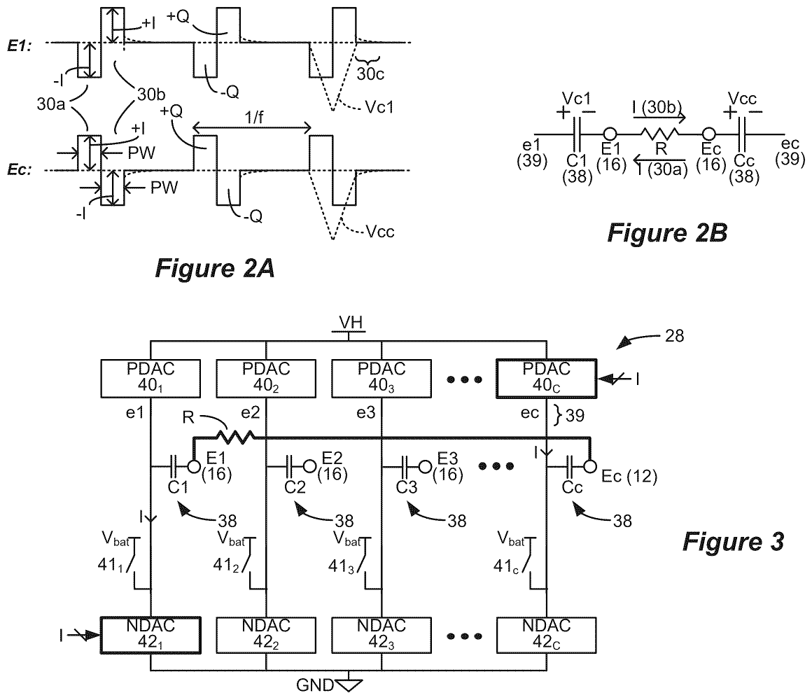

Stimulation in IPG 10 is typically provided by pulses each of which may include a number of phases such as 30a and 30b, as shown in the example of FIG. 2A. The illustrated current is biphasic (i.e., having both an anodic phase (+I) and a cathodic phase (−I)). Stimulation may also be monophasic. In the example shown, such stimulation is monopolar, meaning that a current is provided between at least one selected lead-based electrode (e.g., E1) and the case electrode Ec 12. Stimulation parameters typically include amplitude (current I, although a voltage amplitude V can also be used); frequency (f); pulse width (PW) of the pulses or of its individual phases such as 30a and 30b; the electrodes 16 selected to provide the stimulation; and the polarity of such selected electrodes, i.e., whether they act as anodes that source current to the tissue or cathodes that sink current from the tissue. The stimulation parameters may also include a fractionalization, which refers to a unique arrangement of current driven to each electrode, for example, expressed as a percentage (fraction) of the total current. For example, 50% of the current may be driven to the electrode E1 and 50% of the current may be driven to the electrode E2. A fractionalization can include anodes, cathodes, and combinations of the two. These and possibly other stimulation parameters taken together comprise a stimulation program that the stimulation circuitry 28 in the IPG 10 can execute to provide therapeutic stimulation to a patient.

In the example of FIG. 2A, electrode E1 has been selected as a cathode (during its first phase 30a), and thus provides pulses which sink a negative current of amplitude −I from the tissue. The case electrode Ec has been selected as an anode (again during first phase 30a), and thus provides pulses which source a corresponding positive current of amplitude +I to the tissue. Note that at any time the current sunk from the tissue (e.g., −I at E1 during phase 30a) equals the current sourced to the tissue (e.g., +I at Ec during phase 30a) to ensure that the net current injected into the tissue is zero. The polarity of the currents at these electrodes can be changed: Ec can be selected as a cathode, and E1 can be selected as an anode, etc.

IPG 10 as mentioned includes stimulation circuitry 28 to form prescribed stimulation at a patient's tissue. FIG. 3 shows an example of stimulation circuitry 28, which includes one or more current sources 40; and one or more current sinks 42ᵢ. The sources and sinks 40; and 42; can comprise Digital-to-Analog converters (DACs), and may be referred to as PDACs 40; and NDACs 42; in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node Ei 39 is connected to an electrode Ei 16 via a DC-blocking capacitor Ci 38, for the reasons explained below. PDACs 40; and NDACs 42; can also comprise voltage sources.

Proper control of the PDACs 40; and NDACs 42; allows any of the electrodes 16 and the case electrode Ec 12 to act as anodes or cathodes to create a current through a patient's tissue, R, hopefully with good therapeutic effect. In the example shown, and consistent with the first pulse phase 30a of FIG. 2A, electrode E1 has been selected as a cathode electrode to sink current from the tissue R and case electrode Ec has been selected as an anode electrode to source current to the tissue R. Thus PDAC 40c and NDAC 421 are activated and digitally programmed to produce the desired current, I, with the correct timing (e.g., in accordance with the prescribed frequency F and pulse width PW). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40; and the electrode nodes ci 39, and between the one or more NDACs 42; and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796. The stimulation circuitries described herein provide multiple independent current control (MICC) (or multiple independent voltage control) to guide the estimate of current fractionalization among multiple electrodes and estimate a total amplitude that provide a desired strength. In other words, the total anodic (or cathodic) current can be split among two or more electrodes and/or the total cathodic current can be split among two or more electrodes, allowing the stimulation location and resulting field shapes to be adjusted. For example, a “virtual electrode” may be created at a position between two physical electrodes by fractionating current between the two electrodes.

Much of the stimulation circuitry 28 of FIG. 3, including the PDACs 40; and NDACs 42ᵢ, the switch matrices (if present), and the electrode nodes e; 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation pulses as shown are biphasic, with each pulse comprising a first phase 30a followed thereafter by a second phase 30b of opposite polarity, as mentioned above. Biphasic pulses are useful to actively recover any charge that might be stored on capacitive elements in the electrode current paths, such as on the DC-blocking capacitors 38. Charge recovery is shown with reference to both FIGS. 2A and 2B. During the first pulse phase 30a, charge will build up across the DC-blocking capacitors Cl and Cc associated with the electrodes E1 and Ec used to produce the current, giving rise to voltages Vel and Vcc which decrease in accordance with the amplitude of the current and the capacitance of the capacitors 38 (dV/dt=I/C). During the second pulse phase 30b, when the polarity of the current I is reversed at the selected electrodes E1 and Ec, the stored charge on capacitors Cl and Cc is actively recovered, and thus voltages Vel and Vcc increase and return to OV at the end of the second pulse phase 30b.

To recover all charge by the end of the second pulse phase 30b of each pulse (Vc1=Vcc=OV), the first and second phases 30a and 30b are charged balanced at each electrode, with the first pulse phase 30a providing a charge of −Q (−I*PW) and the second pulse phase 30b providing a charge of +Q (+I*PW) at electrode E1, and with the first pulse phase 30a providing a charge of +Q and the second pulse phase 30b providing a charge of −Q at the case electrode Ec. In the example shown, such charge balancing is achieved by using the same pulse width (PW) and the same amplitude (|I|) for each of the opposite-polarity pulse phases 30a and 30b. However, the pulse phases 30a and 30b may also be charged balanced at each electrode if the product of the amplitude and pulse widths of the two phases 30a and 30b are equal, or if the area under each of the phases is equal, as is known.

FIG. 3 shows that stimulation circuitry 28 can include passive recovery switches 41ᵢ, which are described further in U.S. Patent Application Publications 2018/0071527 and 2018/0140831. Passive recovery switches 41; may be attached to each of the electrode nodes ei 39, and are used to passively recover any charge remaining on the DC-blocking capacitors Ci 38 after issuance of the second pulse phase 30b—i.e., to recover charge without actively driving a current using the DAC circuitry. Passive charge recovery can be prudent, because non-idealities in the stimulation circuitry 28 may lead to pulse phases 30a and 30b that are not perfectly charge balanced.

Therefore, and as shown in FIG. 2A, passive charge recovery typically occurs after the issuance of second pulse phases 30b, for example during at least a portion 30c of the quiet periods between the pulses, by closing passive recovery switches 41ᵢ. As shown in FIG. 3, the other end of the switches 41; not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially decaying curves during 30c in FIG. 2A, which may be positive or negative depending on whether pulse phase 30a or 30b have a predominance of charge at a given electrode.

Passive charge recovery 30c may alleviate the need to use biphasic pulses for charge recovery, especially in the DBS context when the amplitudes of currents may be lower, and therefore charge recovery is less of a concern. For example, and although not shown in FIG. 2A, the pulses provided to the tissue may be monophasic, comprising only a first pulse phase 30a. This may be followed thereafter by passive charge recovery 30c to eliminate any charge build up that occurred during the singular pulses 30a.

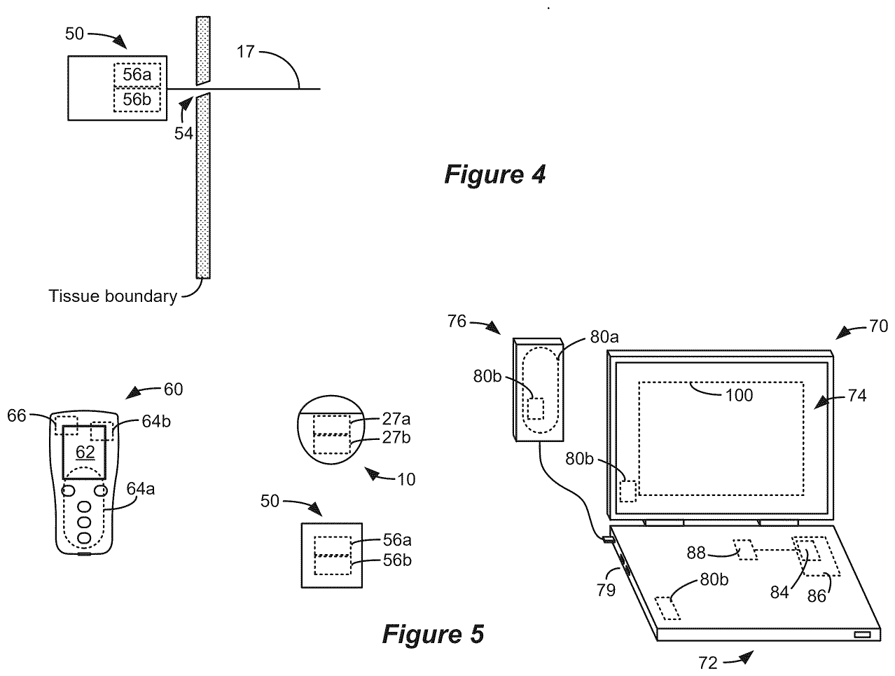

FIG. 4 shows an external trial stimulation environment that may precede implantation of an IPG 10 in a patient, for example, during the operating room to test stimulation and confirm the lead position. During external trial stimulation, stimulation can be tried on the implant patient to evaluate side-effect thresholds and confirm that the lead is not too close to structures that cause side effects. Like the IPG 10, the external trial stimulator (ETS) 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include stimulation circuitry able to form stimulation in accordance with a stimulation program, which circuitry may be similar to or comprise the same stimulation circuitry 28 (FIG. 3) present in the IPG 10. ETS 50 may also include a battery (not shown) for operational power. As the IPG may include a case electrode, an ETS may provide one or more connections to establish similar returns; for example, using patch electrodes. Likewise, the ETS may communicate with the clinician programmer (CP) so that the CP can process the data as described below.

FIG. 5 shows various external devices that can wirelessly communicate data with the IPG 10 or ETS 50, including a patient hand-held external controller 60, and a clinician programmer (CP) 70. Both of devices 60 and 70 can be used to wirelessly transmit a stimulation program to the IPG 10 or ETS 50—that is, to program their stimulation circuitries to produce stimulation with a desired amplitude and timing described earlier. Both devices 60 and 70 may also be used to adjust one or more stimulation parameters of a stimulation program that the IPG 10 is currently executing. Devices 60 and 70 may also wirelessly receive information from the IPG 10 or ETS 50, such as various status information, etc.

External controller 60 can be as described in U.S. Patent Application Publication 2015/0080982 for example and may comprise a controller dedicated to work with the IPG 10 or ETS 50. External controller 60 may also comprise a general-purpose mobile electronics device such as a mobile phone which has been programmed with a Medical Device Application (MDA) allowing it to work as a wireless controller for the IPG 10 or ETS, as described in U.S. Patent Application Publication 2015/0231402. External controller 60 includes a user interface, preferably including means for entering commands (e.g., buttons or selectable graphical elements) and a display 62. The external controller 60's user interface enables a patient to adjust stimulation parameters, although it may have limited functionality when compared to the more-powerful clinician programmer 70, described shortly.

The external controller 60 can have one or more antennas capable of communicating with the IPG 10. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer graphical user interface (GUI) 100 provided on the display 74 of the computing device 72. As one skilled in the art understands, the GUI 100 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. For example, control circuitry 88 can comprise an i5 processor manufactured by Intel Corp, as described at https://www.intel.com/content/www/us/en/products/processors/core/i5-processors.html. Such control circuitry 88, in addition to executing the clinician programmer software 84 and rendering the GUI 100, can also enable communications via antennas 80a or 80b to communicate stimulation parameters chosen through the GUI 100 to the patient's IPG 10.

The user interface of the external controller 60 may provide similar functionality because the external controller 60 can include similar hardware and software programming as the clinician programmer. For example, the external controller 60 includes control circuitry 66 similar to the control circuitry 88 in the clinician programmer 70 and may similarly be programmed with external controller software stored in device memory.

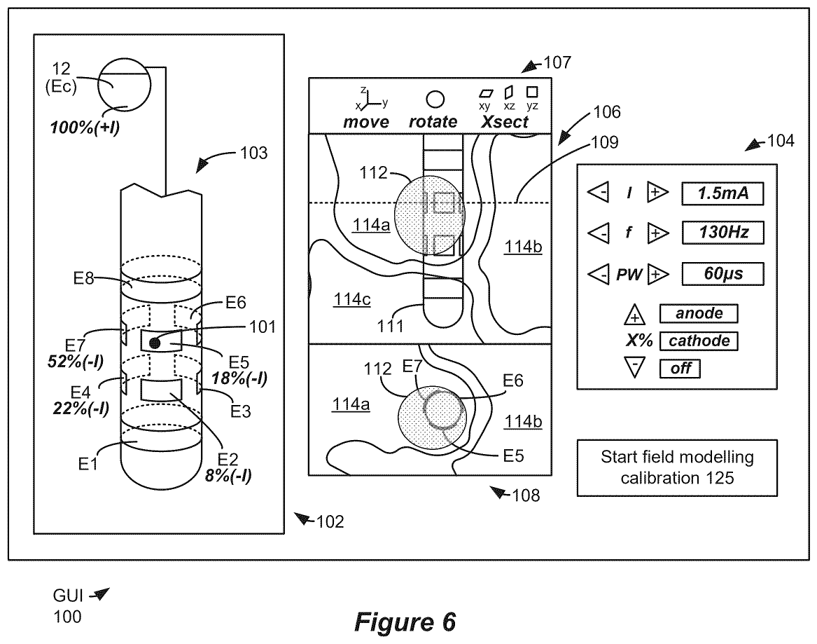

Particularly in the DBS context, it can be useful to provide a clinician with a visual indication of how stimulation selected for a patient will interact with the tissue in which the electrodes are implanted. This is illustrated in FIG. 6, which shows a Graphical User Interface (GUI) 100 operable on an external device capable of communicating with an IPG 10 or ETS 50. Typically, and as assumed in the description that follows, GUI 100 would be rendered on a clinician programmer 70 (FIG. 5), which may be used during surgical implantation of the leads, or after implantation when a therapeutically useful stimulation program is being chosen for a patient. However, GUI 100 could be rendered on a patient external programmer 60 (FIG. 5) or any other external device capable of communicating with the IPG 10 or ETS 50.

GUI 100 allows a clinician (or patient) to select the stimulation program that the IPG 110 or ETS 50 will provide. In this regard, the GUI 100 may include a stimulation parameter interface 104 where various aspects of the stimulation program can be selected or adjusted. For example, interface 104 allows a user to select the amplitude (e.g., a current I) for stimulation; the frequency (f) of stimulation pulses; and the pulse width (PW) of the stimulation pulses. Stimulation parameter interface 104 can be significantly more complicated, particularly if the IPG 10 or ETS 50 supports the provision of stimulation that is more complicated than a repeating sequence of pulses. See, e.g., U.S. Patent Application Publication 2018/0071513. Nonetheless, interface 104 is simply shown for simplicity in FIG. 6 as allowing only for amplitude, frequency, and pulse width adjustment. Stimulation parameter interface 104 may include inputs to allow a user to select whether stimulation will be provided using biphasic (FIG. 2A) or monophasic pulses, and to select whether passive charge recovery will be used, although again these details aren't shown for simplicity.

Stimulation parameter interface 104 may further allow a user to select the active electrodes—i.e., the electrodes that will receive the prescribed pulses. Selection of the active electrodes can occur in conjunction with a leads interface 102, which can include an image 103 of the one or more leads that have been implanted in the patient. Although not shown, the leads interface 102 can include a selection to access a library of relevant images 103 of the types of leads that may be implanted in different patients.

In the example shown in FIG. 6, the leads interface 102 shows an image 103 of a single split-ring lead like that described earlier with respect to FIG. 1B. The leads interface 102 can include a cursor 101 that the user can move (e.g., using a mouse connected to the clinician programmer 70) to select an illustrated electrode 16 (e.g., E1-E8, or the case electrode Ec). Once an electrode has been selected, the stimulation parameter interface 104 can be used to designate the selected electrode as an anode that will source current to the tissue, or as a cathode that will sink current from the tissue. Further, the stimulation parameter interface 104 allows the amount of the total anodic or cathodic current +I or −I that each selected electrode will receive to be specified in terms of a percentage, X. For example, in FIG. 6, the case electrode 12 Ec is specified to receive X=100% of the current I as an anodic current+I. The corresponding cathodic current −I is fractionalized between electrodes E5 (0.18*−I), E7 (0.52*−I), E2 (0.08*−I), and E4 (0.22*−I). Thus, two or more electrodes can be chosen to act as anodes or cathodes at a given time using MICC (as described above), allowing the electric field in the tissue to be steered. The currents specified at the selected electrodes can be those provided during a first pulse phase (if biphasic pulses are used), or during an only pulse phase (if monophasic pulses are used).

GUI 100 can further include a visualization interface 106 that can allow a user to view an indication of the effects of stimulation, such as a stimulation field model (SFM) 112 (also referred to herein as a volume of tissue activated (VTA)) formed using the selected stimulation parameters. The SFM 112 is formed by field modelling, for example, in the clinician programmer 70. The illustrated embodiment of the GUI 99 includes a selection option 125 for initiating such modeling. Only one lead is shown in the visualization interface 106 for simplicity, although again a given patient might be implanted with more than one lead. Visualization interface 106 provides an image 111 of the lead(s) which may be three-dimensional.

The visualization interface 106 preferably, but not necessarily, further includes tissue imaging information 114 taken from the patient, represented as three different tissue structures 114a, 114b and 114c in FIG. 6 for the patient in question, which tissue structures may comprise different areas of the brain for example. Such tissue imaging information may comprise a Magnetic Resonance Image (MRI), a Computed Tomography (CT) image or other type of image. Often, one or more images, such as an MRI, CT, and/or a brain atlas are scaled and combined in a single image model. The visualization interface may also display three dimensional models of relevant regions and/or neurological structures within the brain, including regions that the clinician may wish to stimulate and/or regions that the clinician may wish to avoid. These aspects allow the clinician programmer 70 on which GUI 100 is rendered to overlay the lead image 111 and the SFM 112 with the tissue imaging information in the visualization interface 106 so that the position of the SFM 112 relative to the various tissue structures 114i can be visualized. Images of the patient's tissue may also be taken after implantation of the lead(s), or tissue imaging information may comprise a generic image pulled from a library which is not specific to the patient in question, in some embodiments.

The various images shown in the visualization interface 106 (i.e., the lead image 111, the SFM 112, and the tissue structures 114i) can be three-dimensional in nature, and hence may be rendered in the visualization interface 106 in a manner to allow such three-dimensionality to be better appreciated by the user, such as by shading or coloring the images, etc. Additionally, a view adjustment interface 107 may allow the user to move or rotate the images, using cursor 101 for example.

GUI 100 can further include a cross-section interface 108 to allow the various images to be seen in a two-dimensional cross section. Specifically, cross-section interface 108 shows a particular cross section 109 taken perpendicularly to the lead image 111 and through split-ring electrodes E5, E6, and E7. This cross section 109 can also be shown in the visualization interface 106, and the view adjustment interface 107 can include controls to allow the user to specify the plane of the cross section 109 (e.g., in XY, XZ, or YZ planes) and to move its location in the image. Once the location and orientation of the cross section 109 is defined, the cross-section interface 108 can show additional details. For example, the SFM 112 can allow the user to get a sense of the strength and reach of the stimulation at different locations. Although GUI 100 includes stimulation definition (102, 104) and imaging (108, 106) in a single screen of the GUI, these aspects can also be separated as part of the GUI 100 and made accessible through various menu selections, etc.

Especially in a DBS application, it is important that correct stimulation parameters be determined for a given patient. Improper stimulation parameters may not yield effective relief of a patient's symptoms or may cause unwanted side effects. To determine proper stimulation, a clinician typically uses a GUI such as GUI 100 to try different combinations of stimulation parameters, which generate different SFMs/VTAs. This may occur, at least in part, during a DBS patient's surgery when the leads are being implanted. Such intra-operative determination of stimulation parameters can be useful to determine a general efficacy of DBS therapy. However, finalizing stimulation parameters that are appropriate for a given DBS patient typically occurs after surgery after the patient has had a chance to heal, and after the position of the leads stabilize in the patient. Thus, the patient will typically present to the clinician's office to determine (or further refine) optimal stimulation parameters during a programming session.

Gauging the effectiveness of a given set of stimulation parameters typically involves programming the IPG 10 with that set, and then reviewing the therapeutic effectiveness and side effects that result. Therapeutic effectiveness and side effects are often assessed by one or more different scores(S) for one or more different clinical responses, which are entered into the GUI 99 of the clinician programmer 70 where they are stored with the stimulation parameters set being assessed. Such scores can be subjective in nature, based on patient or clinician observations. For example, bradykinesia (slowness of movement), rigidity, tremor, or other symptoms or side effects, can be scored by the patient, or by the clinician upon observing or questioning the patient. Such scores in one example can range from 0 (best) to 4 (worst).

Scores can also be objective in nature based on measurements taken regarding a patient's symptoms or side effects. For example, a Parkinson's patient may be fitted with a wearable sensor that measures tremors, such as by measuring the frequency and amplitude of such tremors. A wearable sensor may communicate such metrics back to the GUI 99, and if necessary, converted to a score. U.S. Patent Application Publication 2021/0196956, which is incorporated herein by reference in its entirety, discusses determining which symptoms and/or side effects are most sensible to score for a given patient.

This disclosure relates to methods and systems for optimizing stimulation parameters for a DBS patient. In embodiments, the disclosed methods and systems are automated. Disclosed embodiments use patient-specific imaging and/or prior aggregates across a cohort of patients transformed into the patient-specific anatomical space, to find stimulation settings that optimize activating a target region while minimizing the stimulation of regions outside the target that could result in unknown or undesired side effects. This approach utilizes preoperative and/or postoperative neuroimaging data for the patient, paired with postoperative reconstruction of the lead trajectory and three-dimensional SFMs to search the available stimulation space and identify optimized stimulation parameters. In embodiments, three-dimensional models of relevant anatomical or functional structures may be constructed, allowing the clinician to identify target regions/structures and avoidance regions (i.e., regions where stimulation should be avoided). In some embodiments multiple targets and/or multiple avoidance regions may be determined. In some embodiments, weights or priorities may be assigned to the various targets and/or avoidance regions based on the clinician's view of the criticality of stimulating and/or avoiding the respective regions. In other words, the methods and system described herein may determine therapeutic stimulation parameters that meet the above criteria. The therapeutic stimulation set ideally best stimulates the target regions while avoiding the avoidance regions, in consideration to the weights assigned to each.

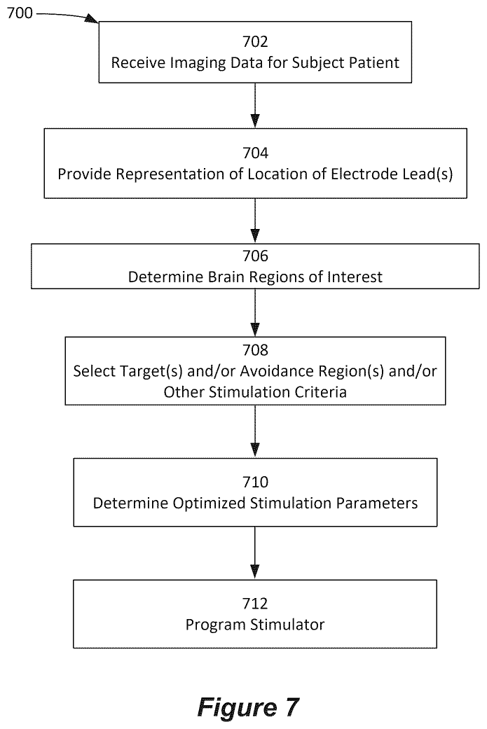

FIG. 7 illustrates a workflow 700 according to some aspects of the disclosure. It is assumed that one or more electrode leads have been implanted within the patient's brain (typically in, or near, a target brain structure) prior to executing the workflow 700. For example, the electrode lead may be implanted in, or near, the patient's STN. Other target brain structures may include the Globus Pallidus, the Ventral Intermediate Nucleus, or any other neural target, as is known in the art. Step 702 involves receiving imaging data for the subject patient. According to some embodiments, the imaging data may include pre-operative MRI data and post-operative CT scans.

Step 704 involves determining/presenting a representation of the electrode lead's position within the patient's brain. According to some embodiments, the location of one or more electrode leads may be determined based on post-implantation clinical CT scans. In some embodiments, the CT scan(s) may be co-registered to the preoperative structural scans, for example, using image fusion algorithms, as is known in the art. An example of an image fusion module is provided in Brainlab Elements (Brainlab, Munich, Germany). The trajectory and orientation of the one or more electrode leads may be extracted, for example, using known algorithms, such as provided in Brainlab Elements or other similar algorithms.

Step 706 involves defining brain regions/features of interest. The brain regions/features of interest may comprise volumes of brain tissue. The regions of interest may correspond to an anatomical structure; anatomical substructure; anatomical or functional region or subregion; any other defined region that is associated with the disease, disorder, condition, symptom, stimulation outcome, or stimulation benefit; any region associated with a physiological signal; or one or more regions that have structural or functional connectivity with another region of interest; or the like or any combination thereof such as aggregation of prior structures or functional regions across a cohort of individuals that are mathematically transformed into the patient specific space. Examples of regions/features of interest may include the STN, the red nucleus (RN), Capsula Interna, Substantia Nigra (SNr), globus pallidus interna (GPi), globus pallidus externa (GPe), sub-regions of any such features, and the like.

According to some embodiments, volumes, models and/or representations of the regions/features of interest may be determined and/or displayed. This may involve processing the image data to determine surfaces for the relevant brain structures. According to some embodiments, three dimensional models of the relevant structures may be generated from fusion of the MRI and CT images using individualized segmentation software auto-segmentation algorithms, as is known in the art. Example algorithms are included in commercial segmentation software, such as Brainlab Elements™, Brainlab, Germany. In embodiments, the models are voxelized, that is, the three-dimensional structure is divided into volume elements, i.e., voxels. Thus, embodiments of the disclosed algorithms provide 3-dimensional voxelized models of the relevant tissues.

Step 708 involves selecting at least one target region of the patient's brain for stimulation and zero or more avoidance region, where stimulation should be avoided. In embodiments, selecting the target and/or avoidance regions entails selecting the corresponding 3-dimension voxelized models of the corresponding regions, which may be displayed on a GUI. Step 708 may also involve selecting one or more stimulation criteria, which in some embodiments, may be considered as bounding parameters for the stimulation. Such stimulation criteria are described below.

According to some embodiments, multiple targets may be selected. For example, the clinician may wish to target the patient's STN to treat cardinal motor symptoms of PD while also targeting the SNr to treat gait disorders, such as freezing of gait. As another example, the clinician may seek to target the patient's GPi to treat cardinal motor symptoms of PD while also targeting the GPe to treat sleep disorders, such as insomnia. According to some embodiments, weights may be associated with target(s), the weights being indicative of the importance or criticality of stimulating the respective target(s).

According to some embodiments, multiple avoidance regions may be selected. A particular aspect of this disclosure concerns allowing the user/clinician to assign (or calculate) different weights for each of the selected targets and/or avoidance regions. The differing weights may be assigned (or calculated) based on a balance between providing more effective therapy by stimulating the target regions and the ability of the patient to tolerate some side-effects caused by the stimulation of the avoidance regions. Likewise, side-effects caused by stimulating some avoidance regions may be more tolerable than side-effects caused by stimulating other avoidance regions. Avoidance regions associated with more tolerable side-effects may be weighted differently than avoidance regions associated with less tolerable side-effects.

For example, consider a situation in which the clinician wishes to target the patient's GPi. Relevant avoidance regions might include the optic tract, which may be associated with visual side effects; the internal capsule, which may be associated with tonic muscle contractions; and the anterior GPi, which may be associated with cognitive and emotional side-effects. Assume that the highest priority is avoiding visual side-effects, the medium priority is avoiding muscle contractions, and the lowest priority is avoiding cognitive effects. Then the optic tract may be weighted highly, the internal capsule weighted medium, and the anterior GPi weighted low. Other examples where weights of various avoidance regions may be used to good effect will be apparent to those of skill in the art.

As mentioned above, Step 708 may also involve selecting one or more stimulation criteria/bounding parameters for the stimulation. Such stimulation criteria may include aspects of the stimulation that the clinician wishes to control or constrain. Examples of stimulation criteria include, but are not limited to, threshold values (e.g., minimum value or maximum value or both to provide a range) for one or more of the following: total charge injected into the tissue, charge injected into the tissue per stimulation pulse or period, total energy delivered, total amplitude of the stimulation, total volume of the stimulated tissue, a level of clinical response from the patient, a limit on one or more side effects (including, but not limited to, mood-related side effects that may be slower to occur), a limit on the volume or percentage of a side effect or avoidance region that is stimulated, a limit on the volume or percentage of the target that is stimulated, or the like or any combination thereof. Other examples of stimulation criteria include, but are not limited to, selecting monopolar stimulation (e.g., monopolar cathodic stimulation or monopolar anodic stimulation), bipolar stimulation, or any other specified multipolar stimulation arrangement; selecting a particular electrode or set of electrodes to be used for stimulation or eligible for use in stimulation; or the like or any combination thereof. In at least some embodiments, an estimated processing time or complexity of the search may be presented to the user. In at least some embodiments, the system may allow the user to elect to not proceed or may elect to adjust the inputs to reduce the processing time or complexity.

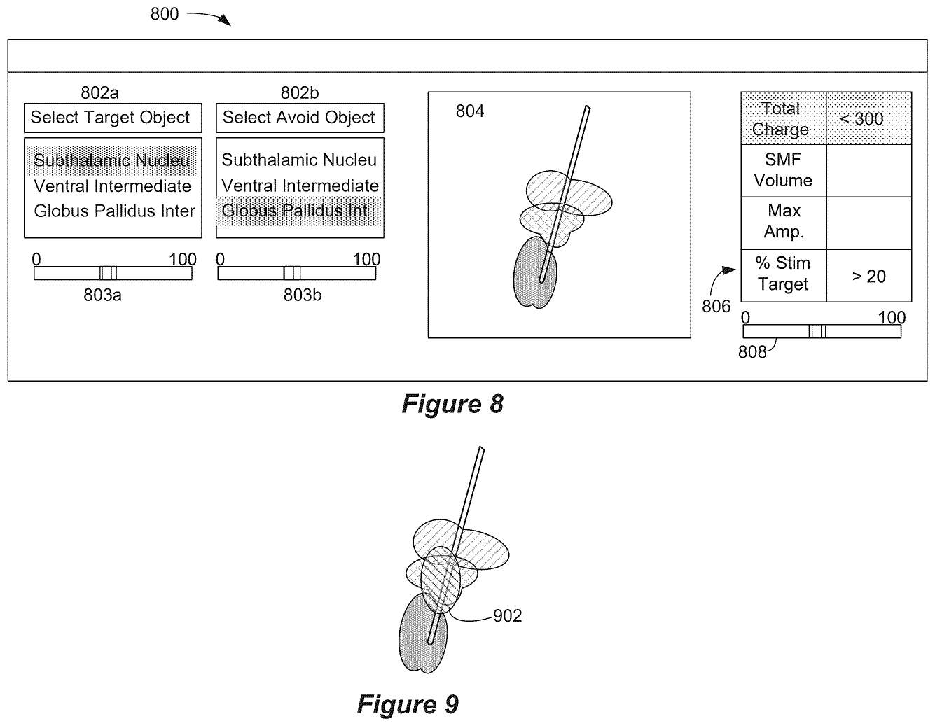

FIG. 8 illustrates an embodiment of a GUI 800 having drop-down elements 802a and 802b that the clinician may use to select target and avoidance regions, respectively. These structures may be created from a patient imaging through a process such as automated segmentation as described above, they may be created by refined segmented anatomy, or could be manually created by the clinician, or could be target volumes imported from some other workflow. In some cases, targets have a basis in anatomy and/or in physiology. The GUI may also include sliders 803a and 803b (or other GUI elements) for assigning weights to the selected targets and/or avoidance regions. The GUI may also be configured to display 804 a representation of the relevant target/avoidance regions determined as described above. In the illustration, the display 804 also shows a representation of a stimulation lead, as described above. The GUI 800 may also be configured to display GUI elements 806 whereby the user can select criteria/bounding parameters as described above. The illustrated GUI also includes a slider 808 to rank the various criteria/bounding parameters. For example, in the embodiment illustrated in FIG. 8, the user has selected “Total Charge” as a criteria/bounding parameter and does not wish the charge to exceed 300 Coulombs. The user may use the slider 808 to set how strongly the algorithm weights the Total Charge bounding parameter. When bounding parameters are selected, the parameter optimization algorithm(s) may be constrained to only consider stimulation parameters/parameter sets (and corresponding SFMs) within the bounded domain.

Referring again to FIG. 7, step 710 involves using a metric optimization algorithm to determine stimulation parameters based on the selected target(s), avoidance region(s), and/or stimulation criteria. The goal of the algorithm is to maximize the overlap of the SFM with each of the target region's volume while staying within the clinician-specified constraints, i.e., the avoidance regions and/or stimulation criteria/bounding parameters. The algorithm may also incorporate the cost of increasing the size of the SFM and the cost of overlapping with avoidance regions, in a consideration of the assigned weights.

According to some embodiments, the algorithm comprises an inverse programming algorithm that may be used to automate the selection of optimized stimulation parameters. Given the indication of which target structures to stimulate and which ones to avoid (i.e., avoidance structures), the inverse programming algorithm may use stimulation field models (SFMs) to optimize the stimulation parameters so as to provide an SFM that best overlaps the desired regions and avoids the undesired ones. Specifically, the algorithms determine how current should be fractionalized among the electrodes to provide the optimum SFM to preferentially stimulate the target(s) and avoid the avoidance region(s), in consideration of the respective assigned weights. Additional stimulation parameters, such as (but not limited to) amplitude, pulse-width, pulse rate, pulse polarity, pulse type, or pattern may be considered.

Algorithms for optimizing stimulation programs using SFMs/VTAs and patient-specific atlases and imaging are described in U.S. Pat. Nos. 11,344,732, 11,195,609, 9,411,935, 9,072,905, and 8,958,615, the contents of which are incorporated herein by reference. An example of a commercial algorithm for optimizing stimulation programs using SFMs/VTAs and patient-specific atlases and imaging is Boston Scientific's DBS Illumina 3-D algorithm (Boston Scientific, Valencia, CA, USA).

As mentioned above, the inverse programming algorithm(s) may determine an optimized “fractionalization” of currents for the electrodes. The fractionalization may be expressed as a percentage of the total current provided to each active electrode (see, e.g., FIG. 6). To generate the SFM/VTA associated with each fractionalization, electric fields resulting from the stimulation setting are constructed as finite element models (FEMs), for example, using programs such as COMSOL Multiphysics software (COMSOL Inc., Burlington, MA, USA). The lead body and the neural tissue may be modeled, as known in the art. A multi-resolute mesh may be created to encompass both the lead body and the surrounding tissue, with highest resolution at electrode-tissue interface and higher resolution in a region of interest (ROI) surrounding the electrode array versus the remaining volume. The scalar potentials at the mesh nodes are calculated and the model is solved once per electrode at unit current (1 mA).

The electric field results from a region of interest (ROI) can be interpolated onto a regular grid of model axons that surround the DBS lead. The response to each stimulus can be computed by temporally scaling the potentials along the axon compartments using a waveform modeled on stimulator recordings to estimate the threshold current (‘Ith’, in mA) at which each axon in the grid fires an action potential from quiescence. A machine learning algorithm (Bootstrap Aggregated Random Forest) which takes features of the axon voltage profile as input and estimates axon's response can be trained, for example, on over 100 million axon simulations. Basis files and the trained predictor can be integrated with the anatomical model of the patient. The output current amplitude thresholds for the axon models are iso-surfaced at the selected stimulation current amplitude. The resulting surface can be displayed as the VTA and overlayed with the representation of the patient's anatomy, if desired. FIG. 9 illustrates an example of an VTA 902 corresponding to a particular set of stimulation parameters overlayed over the display of the anatomical structures, as described above.

Embodiments of the reverse programming algorithm may use a metric optimization algorithm, such as Bound Optimization by Quadratic Approximation (BOBQYA). The goal of the algorithm is to maximize stimulation of a target volume while staying within clinician-specified constraints. The algorithm incorporates the cost of increasing the size of the SFM/VTA, the cost of overlapping with avoidance volumes, including possible side effect regions, as well as stimulation safety limits.

The cost function, or metric, for the optimizer, for each fractionalization, comprises a weighted summation of the stimulated volumes for each of the target regions and avoidance regions and the total SFM/VTA (background volume). In the example embodiment, each of the target regions have a positive weight, and the avoidance regions and background volume have negative weights. In some embodiments, the target and avoidance regions can be in the form of probabilistic maps such that with constant weights some portions of the structures could have higher or lower calculated overlap scores. In addition, in some implementations, the weights can be unequally distributed throughout the volume of each structure, such that overlapping with some portions results in higher or lower calculated overlap scores than others. For each fractionalization, the highest possible metric value is calculated, and the corresponding amplitude is determined.

Equation 1 illustrates an embodiment of an equation to calculate an optimized metric (m) when a single target region is selected, and a plurality of avoidance regions are selected:

m=Σ(vₜ−Σ(v_{a,i}*w_{a,i})−(v_{SFM}*w_{B})) (1)
Where:
m=metric value,
vₜ=stimulated target volume/region,
v_{a, i}=stimulated avoidance volume of the i^{th} avoidance volume/region,
w_{a, i}=weight assigned to the i^{th} avoidance volume,
v_{SFM}=total SFM volume, and
w_{B}=is referred to herein as a “background weight” and is the weight assigned to the cost of generating the SFM.

In Equation 1, the metric is the sum of the stimulated target volume (for example, in mm³) minus the sum of the stimulated volume of each of the avoidance regions, wherein each avoidance region is weighted according to its assigned weight, minus the total volume of the SFM, which may also be weighted with an assigned background weight. Equation 1 includes only a single target volume term vₜ. However, multiple target volumes may be defined, and each may be assigned their own respective weights. The stimulated volumes of the target and avoidance regions may be determined as an overlap of the SFM (determined as described above), with each of the respective regions. Notice that in Equation 1, each of the avoidance regions may be assigned different weights, allowing the clinician to rank or specify the criticality of avoiding the respective regions.

As described above, the user/clinician may select the various weights used in Equation 1, for example, using the sliders 803a and 803b (FIG. 8) or any other input element, as will be apparent to those of skill in the art. The weights represent the cost associated with stimulating the target region balanced against other factors, such as stimulation outside the target region or the generation of side effects. Thus, the weights may be considered as “cost factors.” For example, the v_{SEM} term accounts for the cost (reduction in metric value) associated with generating an SFM of a given volume. The background weight w_{B} may represent a ratio of the cost of stimulating tissue (either in general or outside the target) over the benefit for stimulating the target. Thus, the wp term may be considered as a “background ratio.” Similarly, the undesirability of stimulating each of the respective avoidance regions can be balanced against the desirability of stimulating more of the target region. Thus, the w_{a, i} terms may be indicative of an “avoidance ratio” or “side effects ratio,” i.e., a cost for stimulating a unit volume of tissue in the respective avoidance region over the benefit for stimulating a unit volume of the target. Embodiments of the algorithm may consider other cost parameters, such as a cost parameter based on changing a stimulation parameter, such as amplitude, battery drain, and the like. In some embodiments, the algorithm may consider cost parameters associated with various stimulation criteria, such as the criteria discussed above, and weights associated with those criteria. U.S. Patent Application Publication 2023/0264025, which is incorporated herein by reference in its entirety, discusses deriving cost parameters using stimulation criteria.

The optimization algorithm may be run once for each of two Virtual Electrode (see below) types (one equivalent to the ring electrodes on the lead, and one equivalent to the segmented/directional electrodes on the lead, but with arbitrary placement and rotation). First, the optimizer is run using the ring virtual electrode, and a best solution is determined. If the lead is directional, the optimizer is run using the directional virtual electrode. As the optimization algorithm tests each virtual electrode's position, the position is converted to a fractionalization on the real electrodes of the lead. For each fractionalization, the best metric among the possible amplitudes is compared to the metric of the current best solution. If the new metric is better than the previous best metric, the new metric, virtual electrode type, position, and derived amplitude are stored as the new best solution. When the optimization algorithm has met stop conditions the best solution may be returned and displayed for the clinician.

According to some example embodiments, a Virtual Electrode is a ring (e.g., 1.5 mm height and 360 degrees around the lead) or directional (e.g., 1.5 mm height and 90 degrees around the lead) electrode that, for the calculation of the electrode's voltage field, is modeled as the only electrode on a lead of infinite length with the same nominal lead diameter and material as a real lead. The Virtual Electrode's voltage field is rotated around the axis of the lead and translated along the axis of the lead to model the placement of the virtual electrode at some arbitrary location along and around the active length of the lead. Least-squares fitting is used to determine the fractionalization on the real electrodes that would produce the best fit between the voltage field generated by the real electrodes on the real lead and the voltage field of the virtual electrode placed at the selected location.

The reverse programming methodology described above may be used to determine optimized stimulation parameters based on the metric. It should be noted that the metric may also be used to simply select the best set of stimulation parameters from among a pre-defined or trial sets of stimulation parameters.

As mentioned above, aspects of the disclosure relate to optimizing stimulation parameters to provide DBS stimulation to multiple targets within the patient's brain. According to some embodiments, this may be accomplished by using the algorithm to solve for each target and create an area for each target.

Alternatively, according to some embodiments, optimizing stimulation parameters to optimize multiple targets may comprise using the method described above to find optimized stimulation parameters for each target individually and then combining the solutions' amplitudes and fractionalizations into one amplitude and fractionalization. For example, assume that there are three targets—A, B, and C. The optimized solution for each of the targets will comprise a fractionalization (i.e., F_{A}, F_{B}, and F_{C}) and an amplitude (i.e., Amp_{A}, Amp_{B}, and Amp_{C}). Each combination of a fractionalization and amplitude constitutes a vector. The vectors may be summed in 3D space to create a single vector. The reverse programming methodology described above may be used to map the 3D vector to a real fractionalization and amplitude on the physical lead.

Another embodiment may comprise using the algorithm to solve for each target individually, solve for various combinations of targets, and then combine the solutions in various ways. For example, Equations 2-4 are examples of metrics used to solve for targets A, B, and C, respectively. Equations 5-7 are examples of metrics for the combination (AB), (AC), and (BC), respectively. Equation 8 is an example of a metric for the combination (ABC).

$${m}_{A} = \sum ( {v}_{Α} - \sum ( {v}_{a , i} * {w}_{a , i} ) - ( {v}_{SFM} * {w}_{B} ) ) \qquad ( 2 )$$

$${m}_{B} = \sum ( {v}_{B} - \sum ( {v}_{a , i} * {w}_{a , i} ) - ( {v}_{SFM} * {w}_{B} ) ) \qquad ( 3 )$$

$${m}_{C} = \sum ( {v}_{C} - \sum ( {v}_{a , i} * {w}_{a , i} ) - ( {v}_{SFM} * {w}_{B} ) ) \qquad ( 4 )$$

$${m}_{AB} = \sum ( {v}_{A} + {v}_{B} - \sum ( {v}_{a , i} * {w}_{a , i} ) - ( {v}_{SFM} * {w}_{B} ) ) \qquad ( 5 )$$

$${m}_{AC} = \sum ( {v}_{A} + {v}_{C} - \sum ( {v}_{a , i} * {w}_{a , i} ) - ( {v}_{SFM} * {w}_{B} ) ) \qquad ( 6 )$$

$${m}_{BC} = \sum ( {v}_{B} + {v}_{C} - \sum ( {v}_{a , i} * {w}_{a , i} ) - ( {v}_{SFM} * {w}_{B} ) ) \qquad ( 7 )$$

$${m}_{ABC} = \sum ( {v}_{A} + {v}_{B} + {v}_{C} - \sum ( {v}_{a , i} * {w}_{a , i} ) - ( {v}_{SFM} * {w}_{B} ) ) \qquad ( 8 )$$

In some embodiments, the stimulated target volumes may be assigned weights, though that is not shown in Equations 2-8, for simplicity. Combinations of the metrics, e.g., [(A)+(B)+(C)], [(A)+(BC)], [(B)+(AC)], [(C)+(AB)], and [(ABC)] may be compared to optimize a set of stimulation parameters with the best metric, battery life, etc.

A person of skill in the art will appreciate that the algorithms described above can be used to determine, or facilitate the determination of, stimulation parameters for providing DBS to a particular patient. In some embodiments, one or more target regions may be identified and at least two avoidance regions may be identified. Each of the avoidance regions may be weighted or prioritized differently. In some embodiments, multiple targets may be identified. In multi-target scenarios, one or more avoidance regions may be identified. The avoidance regions and/or the target regions may be weighted differently. The stimulation parameters may be configured to treat any disease, disorder, or symptom that is treatable using DBS. Examples include Parkinson's disease, depression (e.g., treatment-resistant depression), essential tremor, dystonia, epilepsy, obsessive-compulsive disorder, or the like. Referring again to FIG. 7, at step 712, the optimized stimulation parameters may be used to program a stimulator/pulse generator, such as IPG 10 (FIG. 1A).

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method executed on an external programmer for programming a pulse generator (PG) for providing deep brain stimulation (DBS) to a patient having one or more electrode leads implanted in the patient's brain, wherein each electrode lead comprises a plurality of electrodes, the method comprising:
receiving from a user interface (UI) of the external programmer an indication of at least one target region in the patient's brain to be stimulated,
receiving from the UI an indication of two or more avoidance regions in the patient's brain for which stimulation is to be preferentially avoided,
receiving from the UI an indication of weights assigned to each of the two or more avoidance regions, wherein at least two of the weights are different,
for each of a plurality of trial stimulation parameter sets, using control circuitry of the external programmer to:
determine a volume of the at least one target region that will be stimulated by electrical stimulation using the trial parameter set,
determine a volume of each of the avoidance regions that will be stimulated by electrical stimulation using the trial parameter set,
determine a metric based the volumes and the weights,
using the control circuitry to use the metrics to select a therapeutic stimulation parameter set from the plurality of trial stimulation parameter sets, and
using the control circuitry to program the PG with the therapeutic stimulation parameter set.

2. The method of claim 1, wherein determining a volume of the at least one target region that will be stimulated and the volume of each of the avoidance regions that will be stimulated comprises:
determining a stimulation field model (SFM) for the trial stimulation set, wherein the SFM indicates of a volume of tissue activated (VTA) by the electrical stimulation using the parameter set, and
determining a volume of overlap of the SFM with each of the one or more target regions and each of the two or more avoidance regions.

3. The method of claim 2, wherein the metric comprises a value determined by:
(i) determining a weighted overlap for each of the two or more avoidance regions, each weighted overlap comprising the volume of overlap of the SFM with the avoidance region multiplied by the avoidance region's assigned weight,
(ii) summing the weighted overlaps, and
(iii) subtracting a summed overlaps from the volume of overlap of the SFM with one or more target regions.

4. The method of claim 3, wherein the metric is determined using the formula:
$$m = \sum ( {v}_{t} - \sum ( {v}_{a , i} - {w}_{a , i} ) - ( {v}_{SFM} * {w}_{B} ) )$$
wherein m is the metric value, vₜ is the overlap of the SFM with the one or more target regions, v_{a, i} is a volume of overlap of the SFM with an i^{th} avoidance region, w_{a, i} is the weight assigned to the i^{th} avoidance region, v_{SFM} is a total volume of the SFM and w_{B} is a weight assigned to v_{SFM}.

5. The method of claim 2, wherein determining the volume of overlap of the SFM with each of the one or more target regions and each of the two or more avoidance regions comprises:
voxelizing the SFM,
determining 3-dimensional models for each of the one or more target regions and each of the two or more avoidance regions,
voxelizing the 3-dimensional models, and
identifying a number of voxels that are common with the voxelized SFM and each of the one or more target regions and each of the two or more avoidance regions.

6. The method of claim 5, wherein each of the 3-dimensional models are based on imaging of the patient's brain.

7. The method of claim 1, wherein each of the parameter sets comprises a current fractionalization comprising a unique arrangement of current driven to each of the plurality of electrodes.

8. The method of claim 1, wherein each of the two or more avoidance regions are regions, the electrical stimulation of which, are associated with side effects.

9. The method of claim 1, wherein the weight assigned to each of the avoidance regions is indicative of a criticality of avoiding stimulation of the respective avoidance region.

10. The method of claim 1, further comprising:
receiving from the UI an indication of one or more stimulation criteria, and
using the control circuitry to use the one or more stimulation criteria, along with the one or more target regions and the two or more avoidance regions, to select the therapeutic stimulation parameter set.

11. The method of claim 10, wherein the one or more stimulation criteria are selected from the group consisting of a threshold value of one or more stimulation parameters, a total charge injected into the tissue during stimulation, a charge injected into the tissue per stimulation pulse or period, a total energy delivered, a total amplitude, a maximum power usage, and a total volume of the stimulated tissue.

12. The method of claim 10, wherein using the control circuitry to use the one or more stimulation criteria, along with the one or more target regions and the two or more avoidance regions, to select the therapeutic stimulation parameter set comprises eliminating parameter sets that do not meet the stimulation criteria from the plurality of trial stimulation parameter sets.

13. The method of claim 10, further comprising receiving from the UI an indication of weights assigned to each of the one or more stimulation criteria.

14. The method of claim 13, wherein the metric is further determined based on the one or more stimulation criteria their respective weights.

15. The method of claim 1, wherein the therapeutic parameter set is configured to treat one or more of Parkinson's disease, depression (e.g., treatment-resistant depression), essential tremor, dystonia, epilepsy, and/or obsessive-compulsive disorder.

16. The method of claim 1, further comprising displaying one or more selection elements on the UI, whereby a user may select the at least one target region and the two or more avoidance regions.

17. The method of claim 1, further comprising displaying one or more selection elements on the UI, whereby a user may assign weights to the at least one target region and the two or more avoidance regions.

18. The method of claim 1, further comprising using the UI to display one or more images representative of the one or more target regions and the at least two avoidance regions.

19. The method of claim 18, wherein the control circuitry is configured to form the images from one or more of pre-operative and/or post-operative tissue imaging.

20. The method of 18, further comprising using the UI to display a representation of the one or more electrode leads overlaid with the images representative of the one or more target regions and the at least two avoidance regions.
